(19) United States
(12) Patent Application Publication
(10) Pub. No.: US 2007/0060617 A1
(43) Pub. Date: Mar. 15, 2007

(54) 2-Methoxy-5(5-trifluoromethyl-tetrazol-1-yl-benzyl)-2s-phenyl-piperdin-3s-yl)-amine for the treatment of social phobia

(76) Inventor: Massimo Bani, Verona (IT)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/597,792

(22) PCT Filed: Feb. 10, 2005
(86) PCT No.: PCT/EP05/01441

§ 371 Date: Aug. 8, 2006

(30) Foreign Application Priority Data
Feb. 12, 2004 (GB) 0403149.8

(51) Int. Cl. A61K 31/454

(52) U.S. Cl. 514/326

(57) ABSTRACT

The present invention provides the a compound of formula (I) or a pharmaceutically acceptable salt or solvate thereof or pharmaceutical compositions containing the same for the treatment of social phobia (otherwise known as Social Anxiety Disorder).

This invention relates to the use of the compound [2-methoxy-5-(5-trifluoromethyl-tetrazol-1-yl-benzyl)]-([2S,3S]-2-phenyl-piperidin-3-yl)-amine and pharmaceutical compositions containing it in the treatment or prevention of social phobia.

International patent application number WO95/08549 describes novel piperidine derivatives. One such compound described therein is [2-methoxy-5-(5-trifluoromethyl-tetrazol-1-yl-benzyl)]-(2-phenyl-piperidin-3-yl)-amine and it has the following chemical structure (I)

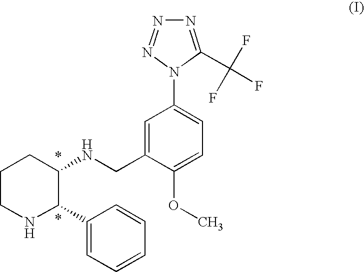

It will be appreciated by those skilled in the art that the compound of formula (I) contains two chiral centres (shown as * in formula (I)) and thus exists in the form of two pairs of optical isomers (i.e. enantiomers) and mixtures thereof including racemic mixtures.

For example, the compound of formula (I) may be either the cis isomer, as represented by figures (a) and (b), or the trans isomer, as represented by figures (c) and (d), or mixtures thereof.

All of the isomers of the compound of formula (I) represented by the figures (a) to (d) and mixtures thereof including racemic mixtures are included within the scope of the invention.

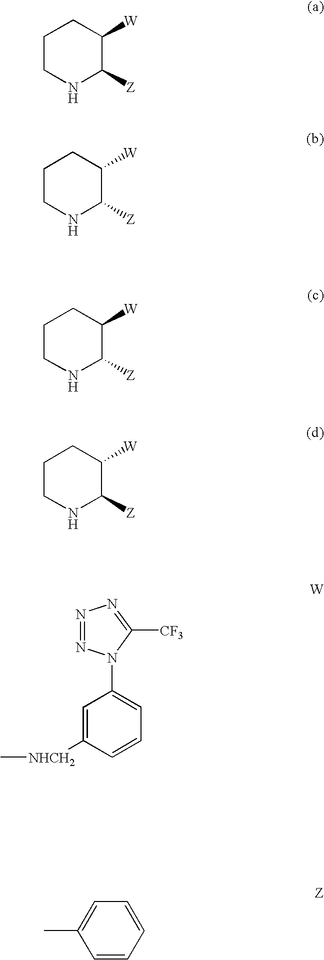

The compound of formula (I) may be in the form of the cis isomer (i.e. as represented by figures (a) and (b)), for example. the 2S, 3S isomer, [2-methoxy-5-(5-trifluoromethyl-tetrazol-1-yl-benzyl)]-([2S,3S]-2-phenyl-piperidin-3-yl)-amine, (i.e. as represented by figure (b)).

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include methanol, ethanol, acetic acid and water. When the solvent is water, the solvate may also be referred to as a hydrate.

It will be appreciated that for use in medicine the salts of formula (I) should be pharmaceutically acceptable. Suitable pharmaceutically acceptable salts will be apparent to those skilled in the art and include for example acid addition salts formed with pharmaceutically acceptable organic or inorganic acids for example, hydrochlorides, hydrobromides, sulphates, alkyl- or arylsulphonates (e.g. methanesulphonates or p-toluenesulphonates), phosphates, acetates, citrates, succinates, tartrates, fumarates and maleates.

Further examples of acid addition salts formed with inorganic acids are e.g. hydrochloric, hydrobromic, sulfuric, nitric or phosphoric acid; and organic acids e.g. succinic, maleic, malic, mandelic, acetic, fumaric, glutamic, lactic, citric, tartaric, benzoic, benzenesulfonic, p-toluenesulfonic, methanesulfonic or naphthalenesulfonic acid. Examples of salts include the hydrochloride, maleate, tosylate or mesylate salts or pharmaceutically acceptable derivatives thereof. Other non-physiologically acceptable salts e.g. oxalates, may be used, for example in the isolation of the compound of formula (I) and are included within the scope of this invention. Also included within the scope of the invention are solvates and hydrates of the compound of formula (I). One such pharmaceutically acceptable salt of the compound of formula (I) for use according to the present invention is the dihydrochloride.

The compound of formula (I) may form acid addition salts with one or more equivalents of the acid. The present invention includes within its scope all possible stoichiometric and non-stoichiometric forms thereof.

Certain salts of the compound of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the salts of compounds represented by formula (I) as mixtures with isomers thereof in which one or more chiral centres are inverted. Likewise, it is understood that salts of compounds of formula (I) may exist in tautomeric forms other than that shown in the formula and these are also included within the scope of the present invention.

The term “pharmaceutically acceptable derivative” as used herein refers to any pharmaceutically acceptable derivative of a compound of the present invention, for example, an ester, which upon administration to a mammal, such as a human, is capable of providing (directly or indirectly) such a compound or an active metabolite thereof. Such derivatives are clear to those skilled in the art, without undue experimentation, and with reference to the teaching of Burger's Medicinal Chemistry And Drug Discovery, 5th Edition, Vol 1: Principles And Practice, which is incorporated herein by reference.

The compound of formula (I) and salts and solvates thereof are described in WO95/08549 as potent and specific NK₁ receptor antagonists.

The compound of formula (I) was initially evaluated for its use in the treatment and prevention of emesis.

The present invention relates to the further use of the compound of formula (I) or a pharmaceutically acceptable salt or solvate thereof in the treatment of social phobia.

Although certain people may experience anxiety when speaking in front of an audience or at other social gatherings, social phobia occurs when this anxiety actually begins to have a large impact on the individual's professional and personal life. This anxiety may manifest itself prior to, as well as during, a social situation. These anxiety symptoms are associated with changes in brain activity and certain receptors in the brain.

Within the context of the present invention, the term ‘social phobia’ (otherwise known as Social Anxiety Disorder) includes various disease states, including Social Phobia, Generalized, and is classified in the Diagnostic and Statistical Manual of Mental Disorders, 4ₜₕ Edition, published by the American Psychiatric Association (DSM-IV), number 300.23. The various forms of the disorders mentioned herein are contemplated as part of the present invention.

In a first aspect thereof, the invention provides the use of the compound of formula (I) or a pharmaceutically acceptable salt or solvate thereof, for the manufacture of a medicament for the treatment of social phobia.

In a further aspect thereof, the invention provides the use of the compound of formula (I) or a pharmaceutically acceptable salt or solvate thereof, for the treatment of social phobia.

In a yet further aspect, the invention provides a method of treatment of social phobia which comprises administering to a human in need thereof an effective amount of the compound of formula (I) or a pharmaceutically acceptable salt or solvate thereof.

In a yet further aspect thereof, the present invention provides a pharmaceutical composition which comprises the compound of formula (I) or a pharmaceutically acceptable salt or solvate thereof for the treatment of social phobia.

Pharmaceutical compositions for use in accordance with the present invention may be formulated in conventional manner using one or more pharmaceutically acceptable carriers or excipients.

It will be appreciated that reference to treatment is intended to include prophylaxis as well as the alleviation of established symptoms.

Thus, the compound of formula (I) and its pharmaceutically acceptable salts and solvates may be formulated for oral, buccal, parenteral, topical (including ophthalmic and nasal), depot or rectal administration or in a form suitable for administration by inhalation or insufflation (either through the mouth or nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the composition may take the form of tablets or formulated in conventional manner.

The compound of formula (I) or its pharmaceutically acceptable salts or solvates may be formulated for parenteral administration by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compound of formula (I) or its pharmaceutically acceptable salts or solvates may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compound of formula (I) or its pharmaceutically acceptable salts or solvates may also be formulated in rectal compositions such as suppositories or retention enemas, e.g. containing conventional suppository bases such as cocoa butter or other glycerides.

The compound of formula (I) or its pharmaceutically acceptable salts or solvates may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compound of formula (I) or its pharmaceutically acceptable salts and solvates may be formulated as solutions for administration via a suitable metered or unitary dose device or alternatively as a powder mix with a suitable carrier for administration using a suitable delivery device.

A proposed dose of the compound of the invention is 1 to about 1000mg per day. It will be appreciated that it may be necessary to make routine variations to the dosage, depending on the age and condition of the patient and the precise dosage will be ultimately at the discretion of the attendant physician or veterinarian. The dosage will also depend on the route of administration and the particular compound selected.

Thus, for parenteral administration a daily dose will typically be in the range of 1 to about 100 mg, such as 1 to 80 mg per day. For oral administration a daily dose will typically be within the range 1 to 100 mg e.g. 10 to 50 mg.

The compound of formula (I) or its pharmaceutically acceptable salts of solvates thereof may be prepared by the process described in international patent application no. WO95/08549, which is incorporated herein by reference.

It will be appreciated by those skilled in the art that the compound of formula (I) or pharmaceutically acceptable salts or solvates thereof according to the invention may advantageously be used in conjunction with one or more other therapeutic agents, for instance, 5-HT uptake inhibitors (such as escitalopram, escitalopram oxalate, venlafaxine, sertraline, fluvoxamine or paroxetine), GABA receptor agonists (such as pregabalin) and monoamine oxidase A inhibitors (such as moclobemide).

Pharmacological Activity

The present invention may be illustrated by suitable patient studies. The following examples of suitable patient studies are for illustrative purposes and are not intended to limit the scope of the invention in any way.

The first study was a double-blind, placebo-controlled study of the effect of [2-methoxy-5-(5-trifluoromethyl-tetrazol-1-yl-benzyl)]-(2-phenyl-piperidin-3-yl)-amine dihydrochloride in patients suffering from social phobia (otherwise known as Social Anxiety Disorder).

Thirty six patients aged between 19 and 48 meeting the DSM-IV criteria for social phobia (as set out in Diagnostic and Statistical Manual of Mental Disorders, 4th Edition, published by the American Psychiatric Association) were selected. Prior to the first Positron Emission Tomography (PET) investigation, patients were marked for severity in triplets, based on the Social Phobia Screening Questionnaire and as far as possible for sex and age. Patients were randomly allocated to one of three groups: NK1-antagonist, selective serotonin reuptake inhibitor (SSRI), or placebo. The NK1-antagonist group received the NK1 antagonist [2-methoxy-5-(5-trifluoromethyl-tetrazol-1-yl-benzyl)]-(2-phenyl-piperidin-3-yl)-amine dihydrochloride (daily oral dose of 5 mg) and the SSRI group received citalopram (daily oral dose of 40 mg).

After six weeks of treatment, the medication was suspended and patients received follow-up assessments at two and four weeks after the treatment period. The effect of [2-methoxy-5(5-trifluoromethyl-tetrazol-1-yl-benzyl)]-(2-phenyl-piperidin-3-yl)-amine dihydrochloride on anxiety symptoms and brain activity in patients diagnosed with social phobia was evaluated using self-report questionnaires (the Social Phobia Scale (SPS), the Social Interaction Anxiety Scale (SIAS), the Personal Report on Confidence as a Speaker (PRCS), the Social Phobia Screening Questionnaire (SPSQ), the Sheehan Disability Inventory (SDI), self-report versions of the Liebowitz Social Anxiety Scale (LSAS-SR) and the Global Assessment of Functioning (GAF) scale), state anxiety measures (the Speilberger state-anxiety inventory (STAI-S), subjective ratings of fear and distress on 0-100 (min-max) scales and heart rate), verbal performance (comparing the number of spoken syllables during the first ten seconds of each videotaped speech) and PET assessments.

Patients with social phobia were significantly improved after short-term treatment with the NK1-antagonist [2-methoxy-5-(5-trifluoromethyl-tetrazol-1-yl-benzyl)]-(2-phenyl-piperidin-3-yl)-amine dihydrochloride or citalopram and both drugs were generally superior to placebo. The clinical and behavioural effect of [2-methoxy-5-(5-trifluoromethyl-tetrazol-1-yl-benzyl)]-(2-phenyl-piperidin-3-yl)-amine dihydrochloride was similar to that of citalopram even though it was administered for a shorter period i.e. four as compared to six weeks.

The second study is a 12-week randomised, multicentre, double-blind, placebo controlled, fixed dose, parallel group study to assess the efficacy, safety and tolerability of [2-methoxy-5-(5-trifluoromethyl-tetrazol-1-yl-benzyl)]-(2-phenyl-piperidin-3-yl)-amine dihydrochloride (5 mg/day) versus placebo in patients with a psychiatric diagnosis of Social Anxiety Disorder (SAD) according to DSM-IV 300.23.

Following an initial screening visit, patients fulfilling the study inclusion and exclusion criteria will enter a one-week single-blind placebo run-in phase to further evaluate their suitability for entry into the study and to eliminate early placebo responders. Eligible patients will be randomised at the baseline visit to either [2-methoxy-5-(5-trifluoromethyl-tetrazol-1-yl-benzyl)]-(2-phenyl-piperidin-3-yl)-amine dihydrochloride or placebo (1:1 ratio) for a twelve week double-blind treatment phase. Placebo patients will receive study medication identical in appearance to that received by patients assigned to the active medication. All patients will be required to return for a follow-up visit 14 days after the last dose of study medication. In addition, all patients with ongoing adverse events at this visit will be required to return for a further follow-up visit within 28 days. The primary efficacy parameter will be the change from baseline in the Liebowitz Social Anxiety Scale (LSAS) total score at the Week 12 endpoint The Liebowitz Social Anxiety Scale (LSAS) will be administered by the investigator.

Male and female outpatients between the ages of 18 to 65 years with a primary diagnosis of Social Anxiety Disorder (DSM-IV, 300.23) will be enrolled into this study. A total of approximately 260 patients will be randomised (130 to each of [2-methoxy-5-(5-trifluoromethyl-tetrazol-1-yl-benzyl)]-(2-phenyl-piperidin-3-yl)-amine dihydrochloride and placebo).

The null hypothesis for this study is that there is no difference between [2-methoxy-5-(5-trifluoromethyl-tetrazol-1-yl-benzyl)]-(2-phenyl-piperidin-3-yl)-amine dihydrochloride and placebo in the change from baseline in the LSAS score at Week 12 LOCF (last observation carried forward) in the intent-to-treat (ITT) population. The alternative hypothesis is that there is a difference between [2-methoxy-5-(5-trifluoromethyl-tetrazol-1-yl-benzyl)]-(2-phenyl-piperidin-3-yl)-amine dihydrochloride and placebo. The null hypothesis will be rejected if there is sufficient evidence that [2-methoxy-5-(5-trifluoromethyl-tetrazol-1-yl-benzyl)]-(2-phenyl-piperidin-3-yl)-amine dihydrochloride is superior to placebo. The ITT (Intention To Treat) population consists of all randomised subjects who received at least one dose of double blind medication and for whom at least one post-baseline assessment is available.

Two analysis populations will be evaluated. The ITT population will consist of all patients who were randomised, received at least one dose of blinded medication and for whom at least one post baseline assessment is available. This will be the primary population of interest. In addition a PP (Per Protocol) population will also be evaluated for the primary efficacy variable. The PP population will be a subset of the ITT population including only those patients who do not violate the protocol to an extent that could compromise the treatment evaluation.

The primary comparison of interest is [2-methoxy-5-(5-trifluoromethyl-tetrazol-1-yl-benzyl)]-(2-phenyl-piperidin-3-yl)-amine dihydrochloride versus placebo for the change from baseline to week 12 LOCF in the LSAS score in the ITT population. This comparison will be made at the two-sided 5% level of significance. Analysis of covariance (i.e. a linear model assuming normal errors) will be used for the primary inference. The model will include terms for centre, baseline LSAS score and treatment group, regardless of their significance. No interaction terms will be included in this primary model. A Hepatic Safety Review Committee (HSRC) will be utilised during the conduct of this study. Liver safety will be monitored during the course of the study with LFT data for all subjects who experience an elevation of ≧2X the upper limit of the reference range being forwarded to the HSRC by the Medical Monitor at least monthly.

1-4. (canceled)

5. The method according to claim 8 wherein the compound is [2-methoxy-5-(5-trifluoromethyl-tetrazol-1-yl-benzyl)]-([2S,3S]-2-phenyl-piperidin-3-yl)-amine or a pharmaceutically acceptable salt or solvate thereof.

6. (canceled)

7. The method according to claim 5 wherein the pharmaceutically acceptable salt is the dichloride.

8. A method of treatment of social phobia which comprises administering to a human in need thereof an effective amount of the compound of formula (I): 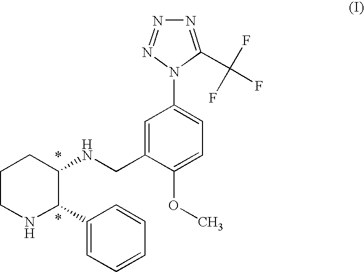
or a pharmaceutically acceptable salt or solvate thereof.
